(19) United States
(12) Patent Application Publication
(10) Pub. No.: US 2006/0223160 A1
(43) Pub. Date: Oct. 5, 2006

(54) Systems and methods for the isolation and identification of microorganisms from hydrocarbon deposits

(75) Inventor: Gary Vanzin, Arvada, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LUCA Technologies, LLC, Denver, CO (US)

(21) Appl. No.: 11/099,879

(22) Filed: Apr. 5, 2005

(51) Int. Cl. C12N 1/20

(52) U.S. Cl. 435/252.4

(57) ABSTRACT

The present invention provides systems and methods to isolate microorganisms and optionally their genetic material. The isolation of microorganisms is conducted under anaerobic or other similar conditions to facilitate the preparation of microorganisms that are anaerobes, whether obligate or facultative, as well as microaerophiles. In an alternative format, the isolation of microorganisms is from a carbonaceous substrate which typically hinders or retards isolation therefrom.

### FIELD OF THE INVENTION

The present invention relates to isolation of microorganisms and/or their genetic material. The isolation of microorganisms is conducted under anaerobic or other similar conditions to facilitate the preparation of microorganisms that are anaerobes, whether obligate or facultative, as well as microaerophiles. In an alternative format, the isolation of microorganisms is from a carbonaceous substrate which typically hinders or retards isolation therefrom.

### BACKGROUND OF THE INVENTION

As world energy demands continues to increase, energy resources are being extracted from sub-surface formations of carbonaceous material (e.g., oil, coal, etc.) at increasing rates. The initial extraction, sometimes referred to a primary recovery, normally requires the least capital and resources. As the easily extracted material is depleted, extraction costs can escalate rapidly, and formations are abandoned in favor of new sites with more easily extractable stocks. An abandoned formation, however, still contains significant amounts of carbonaceous material. The mined coal formation in the Powder River Basin in northeastern Wyoming, for example, is still estimated to contain approximately 1,300 billion short tons of coal. If just 1% of this coal could be economically converted into natural gas, it could supply the current annual natural gas needs of the entire United States (i.e., about 23 trillion cubic feet) for the next four years. Several other mined coal and oil formations of this magnitude are present in the United States.

Of course, even if the remaining carbonaceous materials could be economically extracted in their native state, several products of their combustion, including sulfur compounds (SOₓ), nitrogen compounds (NOₓ), and carbon dioxide (CO₂), are believed to place significant stress on the environment. Concern about the environmental impact of burning fossil fuels has lead to national and international initiatives to develop alternative energy sources that generate lower levels of the pollutants. One such initiative that is receiving considerable government and private sector support is the development of hydrogen engines and fuel cells for vehicle propulsion and electricity generation. The combustion of molecular hydrogen (H₂) into water presents a more benign environmental alternative to burning gasoline, oil or coal.

Unfortunately, hydrogen is more accurately characterized as an energy carrier than a fuel source. Very little molecular hydrogen exists in nature, so another energy source is needed to make the hydrogen, which transports the energy to the site where it will be released by chemical reaction (e.g., combustion). A power and transportation infrastructure based on hydrogen will require plentiful supplies of energy and/or feedstock materials used to make the hydrogen. One well known method of making hydrogen is the steam reforming of methane, where methane (CH₄) and steam (H₂O) are converted into carbon monoxide (CO) and hydrogen (H₂). Accordingly, a robust hydrogen economy will require large sources of light hydrocarbon feedstock materials such as methane.

The above discussion and citation of documents herein is not intended as an admission that any is pertinent prior art. All statements as to the date or representation as to the contents of documents is based on the information available to the applicant and does not constitute any admission as to the correctness of the dates or contents of the documents.

### BRIEF SUMMARY OF THE INVENTION

The present invention relates to the isolation of microorganisms that participate in the degradation of large or complex hydrocarbons found in naturally occurring sources, such as those present in underground formations. The microorganisms are useful for the recovery of energy contained within large or complex hydrocarbons, many of which are associated with other materials that hinder extraction of the hydrocarbons from the formations, by converting the hydrocarbons to smaller molecules that can be more readily recovered or extracted. The isolation of the microorganisms aids in the identification of the particular microorganism that may be present in a given formation as well as provides for the ability to reintroduce an isolated microorganism into a formation to aid in the conversion of hydrocarbons into smaller molecules that are more readily extracted or recovered.

The invention is driven in part on energy recovery by conversion of large or complex hydrocarbons to smaller hydrocarbons, optionally with release thereof from materials that hinder extraction of large or complex hydrocarbons. This approach is based on biogenic conversion of carbonaceous materials in underground formations, which conversion has received relatively little commercial attention. Large potential sources of energy, locked up in carbonaceous materials such as (but not limited to) coal, residual oil, etc., may be more readily recovered by conversion of the hydrocarbons in the carbonaceous materials, as well as the carbonaceous material itself, into methane and other light hydrocarbons. In biogenic conversion, microorganisms treat the carbonaceous materials as a source of raw materials for conversion into smaller, lighter metabolites including alcohols, organic acids, aromatic compounds, molecular hydrogen, and/or methane as non-limiting examples. Conversion by microorganisms includes their reformation or utilization of starting materials to form products by metabolism, including catabolism and/or anabolism by a plurality of microorganisms of differing species. The plurality of microorganisms may be considered a microbial consortium.

Given that in in situ in sub-surface formations, the concentrations of free oxygen (i.e., O₂) often falls below the level that can sustain aerobic metabolism in microorganisms (or strict aerobic microorganisms), it is possible that anaerobic microorganisms (including obligate and/or facultative anaerobic microorganisms or microaerophiles) predominate. Unfortunately, most anaerobic microorganisms cannot survive in the oxygen rich atmosphere above ground, and are difficult to study in conventional laboratories. For this reason and others, anaerobic microorganisms that can metabolize carbonaceous materials are poorly understood. The invention provides for the isolation of a microorganism, alone or as part of a consortium or coculture, from such sub-surface environments. Isolatable microorganisms include those that participate in the biogenic conversion of carbonaceous material, as well as the hydrocarbons therein, into molecules with a higher molar percentage (mol. %) of hydrogen atoms than in the carbonaceous material or hydrocarbons therein. Non-limiting examples of molecules with a high mol. % of hydrogen atoms include molecular hydrogen (H₂) and methane (CH₄).

In a first aspect, the invention provides a method for isolating a microorganism from the environment in which it is naturally found, such as, but not limited to, from a geologic formation comprising other organisms and/or other chemical compounds found in the formation. The formation may be underground or subterranean. The microorganism may be isolated as in pure culture, such that only one species is present, or in the form of a microbial community, coculture, or consortium, comprising a plurality of two or more different species of microorganisms. In some embodiments, an isolated microbial community, coculture, or consortium contains two or more different microorganisms that are metabolically related, such as where the microorganisms have a symbiotic relationship with each other. The invention thus includes methods for isolating a microbial community, coculture or consortium wherein two or more of the species of microorganisms present therein are related by syntrophy such that one microorganism is a syntroph of one or more other microorganisms. Isolation of a microbial community, coculture or consortium is advantageous where individual syntroph microorganisms cannot be separately cultured or propagated (in the absence of the related syntroph(s)). This benefit is of particular relevance in the estimated 99% of cases where an individual microorganism cannot be cultured as a single species culture.

The microorganisms to be isolated are preferably one or more of a subterranean substrate that contains viable microorganisms. The isolation methods of the invention maintain the viability of one or more of the isolated microorganism during the isolation process. The methods may also be considered as preparing a culture of one or more microorganisms.

The microorganism(s) may be isolated as part of a microbial consortium for biogenically increasing the hydrogen content of a carbonaceous source material. In some embodiments, the consortium may include 1) microorganisms capable of converting or metabolizing a carbonaceous source material into a product containing one or more first intermediate hydrocarbons; or 2) microorganisms that include one or more species of Thermotoga or Pseudomonas capable of converting the first intermediate hydrocarbons into a product containing one or more second intermediate hydrocarbons and a molecule containing an oxidized carbon atom; or 3) microorganisms capable of converting the second intermediate hydrocarbons into a product containing one or more smaller hydrocarbons and water. In other embodiments, the smaller hydrocarbons have a greater mol. % of hydrogen atoms than the carbonaceous source material. In addition to isolating a consortium comprising any one of these three groupings of microorganisms, the invention provides for the isolating of any combination of two or more of these groupings.

Alternatively the isolated microorganisms may be capable of biogenically producing methane from a larger hydrocarbon. In some embodiments, the consortium may include 1) microorganisms capable of converting or metabolizing the larger hydrocarbon into a product containing one or more intermediate hydrocarbon compounds; or 2) microorganisms that include one or more species of Pseudomonas or Thermotoga capable of converting the intermediate carbon compounds into a product containing carbon dioxide and molecular hydrogen; or 3) microorganisms capable of converting or metabolizing the carbon dioxide and molecular hydrogen into methane and water. In addition to isolating a consortium comprising any one of these three groupings of microorganisms, the invention provides for the isolating of any combination of two or more of these groupings.

In additional embodiments of the invention, the isolated microorganisms may be in the form of a consortium for anaerobic production of methane from larger hydrocarbons. The consortium may include 1) one or more species of Thermotoga or Pseudomonas capable of converting or metabolizing the larger hydrocarbons to form a product containing smaller hydrocarbons; or 2) microorganisms capable of converting or metabolizing at least a portion of the smaller hydrocarbons to form acetate; or 3) microorganisms capable of converting or metabolizing the acetate to form methane and water. In addition to isolating a consortium comprising any one of these three groupings of microorganisms, the invention provides for the isolating of any combination of two or more of these groupings.

In further embodiments, the isolated microorganism(s) may include a microorganism other than from the genus Pseudomonas. The microorganism may, for example, be a species from the genus of Gelria, Clostridia, Moorella, Thermoacetogenium, Pseudomonas, or Methanobacter or be another species of microorganism with the same capabilities as the microorganisms and/or consortia described herein.

To obtain an anaerobic microorganism or consortium, the invention provides for the use of anaerobic conditions, or an anoxic environment as an alternative, during the isolation process. This may be followed by maintenance of the isolated microorganism(s) or consortium under anaerobic conditions. While anaerobic conditions may be considered as the absence of cellular metabolism with minimal or no use of molecular oxygen as the typical electron acceptor, the conditions may also be defined as being is below that in earth's atmosphere, such as at the troposphere. Alternatively, anaerobic may be defined as being below about 18% free oxygen by mol., including less than about 10%, less than about 5%, less than about 2%, or less than about 0.5% by mol. molecular oxygen in the environment of a microorganism, coculture, or consortium. In embodiments comprising formation water, the level of oxygen may contain less dissolved oxygen than what is typically measured for surface water (e.g., about 16 mg/L of dissolved oxygen). For example, the formation water may contain less than about 14, less than about 12, less than about 10, less than about 8, less than about 6, less than about 4, less than about 2, or less than about 1 mg/L dissolved oxygen.

In alternative embodiments, the environment may be “methanogenic” in that methanogenesis is the typical final electron accepting process in cellular metabolism to produce methane. The invention thus includes the use of conditions without the addition of common exogenous electron acceptors, like oxygen or nitrate or sulfate as non-limiting examples, into the disclosed methods. The lack of such added electron acceptors permits the methods to be advantageously used in the isolation or preparation of microorganism(s) that produce methane.

The environment of a microorganism includes any gas, liquid or solid that may be present where anaerobic conditions would be measured or maintained within each phase (gas, liquid or solid) of the environment. In the case of the gaseous portion of the environment, the invention provides for the use of an inert or relatively inert gas with the microorganism(s). Non-limiting examples include molecular nitrogen, helium, neon, and argon as well as methane and carbon dioxide.

The isolated microorganism(s) of the invention may be introduced into a geological formation to increase, or result in, the production of molecular hydrogen and/or methane due to the presence of the metabolic activities present in the microorganism(s). The introduction maybe accompanied by, preceded by, or followed by, introduction of one or more agents to into the formation to result in conditions, in all or part of the formation, conducive to the growth of the microorganism(s). Of course the isolated microorganism(s) may also be propagated ex situ or in culture under the conditions described herein for their isolation.

In a second aspect, the invention provides for the isolation of one or more microorganisms from a carbonaceous substrate, such as, but not limited to, that of a subterranean or sub-surface formation as described herein. The isolated microorganisms may then be used as a source of genetic material for subsequent use, including 1) the identification of the microorganism(s) present in the carbonaceous substrate and 2) the cloning of microbial sequences as non-limiting examples. The microorganism(s) may be those adsorbed or absorbed onto a surface or subsurface of the substrate. Elution or desorption from the substrate is performed in the presence of one or more anions and a zwitterionic detergent. Mechanical agitation may also be used to facilitate the isolation of the microorganism(s). The genetic material from the isolated microorganism(s) may be subsequently assayed or tested to determine the type of microorganism(s) present in the substrate.

In some embodiments, the first and second aspects of the invention may be used in combination. As a non-limiting example, the isolation of microorganisms, a microbial community, or a coculture (first aspect) may be followed by isolation of microorganisms from carbonaceous material in the culture in accordance with the second aspect.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

FIG. 1 shows a flowchart with method steps for making and measuring the characteristics of a consortia according to embodiments of the invention.

### DETAILED DESCRIPTION OF THE INVENTION

Methods for the isolation of anaerobic consortia are described to obtain cultures of microorganisms from a subterranean substrate or from a laboratory-generated culture mimicking a carbonaceous or hydrocarbon-containing in situ environment of a subterranean substrate of the invention. The methods may be used to isolate obligate and/or facultative anaerobic microorganisms, as well as microaerophiles, depending on the available oxygen used during isolation. The microorganisms may be isolated as pure cultures comprising a single species or as a microbial community, coculture or consortium comprising two or more species, or two or more subtypes of a species, of microorganisms.

In one embodiment, a method of preparing a culture of microorganisms is provided. The method comprises obtaining an anaerobic or anoxic subterranean substrate comprising viable microorganisms; and maintaining the substrate under anaerobic or anoxic conditions to form a culture. The method is practiced under conditions wherein said microorganisms remain viable in said culture. Parameters of the conditions that can be adjusted or modified as desired or necessary include temperature, pH, oxidation potential (Eh), nutrient concentrations, salinity, ionic strength, and metal ion concentrations as non-limiting examples. Of course all materials, including culture media and buffer solutions as non-limiting examples, used in the method are produced under suitable anaerobic or anoxic conditions as known to the skilled person. As used herein, “viable” refers to the ability to maintain cell metabolism or cell growth and/or proliferation, while “anoxic” refers to the the absence of molecular oxygen, such as in cases of anoxic water found in subterranean locations, such as mines and wells from which fossil fuels have been extracted.

In some embodiments, anaerobic conditions are those wherein little or no cellular metabolism occurs with the use of molecular oxygen as the typical electron acceptor. This is often the case in cases of underground or subterranean locations where available molecular oxygen has been consumed by aerobic metabolism. Such conditions may be mimicked or maintained ex situ simply by avoiding or limiting the introduction of molecular oxygen (such as in gaseous or dissolved forms) into the environment of the microorganisms being isolated. In alternative embodiments, the environment may be made “methanogenic” in that methanogenesis is the typical final electron accepting process in cellular metabolism to produce methane. This may be practiced by simply avoiding or limiting the introduction of common exogenous electron acceptors into the environment of the microorganisms being isolated. Of course in cases of a microbial community, coculture, or other cases of multiple microorganisms, there may be “terminal” electron acceptors that are used to produce carbon dioxide or molecular hydrogen as the waste product or “final product” in some microorganisms. But where methanogenesis is available, these “terminal” electron acceptors are used by methanogenic microorganisms (or methanogens) that are present to produce energy by generation of methane as the final electron accepting process.

As used herein, an electron acceptor is a molecule or compound that accepts or receives one or more electrons (from an electron donor) during cellular metabolism or respiration (where “respiration” is not limited to situations where carbon dioxide is formed). Thus the electron acceptor is reduced, and the electron donor is oxidized. Non-limiting examples of an electron acceptor include molecular oxygen, nitrate, iron (III), manganese (IV), sulfate, carbon dioxide, or in some cases the chlorinated solvents such as tetrachloroethene (PCE), trichloroethene (TCE), dichloroethene (DCE), and vinyl chloride (VC). These examples represent commonly used electron acceptors which may be exogenously supplied, or not, to the microorganisms of the invention.

In embodiments lacking the addition of an exogenous electron acceptor, endogenously present or produced electron acceptors would be used. Non-limiting examples of endogenously present or produced electron acceptors include aromatic hydrocarbons as well as polycyclic aromatic acids present with, or produced by the microorganisms of the invention. In some embodiments, these types of electron acceptors may be supplied to microorganisms of the invention. In other embodiments, methanogenesis to produce methane is the process by which a terminal electron accepting event occurs.

Microorganisms that may be isolated by use of the invention include any bacterium (eubacteria) or archaebacterium (archaea, including methanogens, halophiles, and thermophiles) or lower eukaryote (including yeasts, fungi and molds). Archaebacteria are divided into three phyla: methanogens, extreme halophiles, and thermoacidophiles. The methanogens are capable of converting H₂ and CO₂ into methane gas. Extreme halophiles thrive in high salt conditions and may produce metabolites used by other microorganisms. They would be predicted to be found in formations containing high salt concentrations. Thermoacidophiles are found in highly acidic environments with very high temperatures. Temperatures of up to 230° Fahrenheit and pH below 2 have been tolerated by some thermoacidophiles, which would be expect to be found in formations with low pH and high temperatures.

The isolated microorganism(s) may be obligate anaerobes that cannot survive in an atmosphere with molecular oxygen concentrations that approach those found in tropospheric air (e.g., 18% to 21%, by mol. in dry air) or those for which oxygen is toxic. The microorganism(s) may also be facultative aerobes and anaerobes that can adapt to both aerobic and anaerobic conditions. A facultative anaerobe is one which can grow in the presence or absence of oxygen, but may, in some instances, grow better in the presence of oxygen. The microorganism(s) may also include one or more microaerophiles that are viable under reduced oxygen conditions, even if they prefer the absence of oxygen. Some microaerophiles proliferate under conditions of increased carbon dioxide of about 10% mol or more (or above about 375 ppm). Microaerophiles are thought to include at least some species of Thermotoga as well as Thermicanus, Beggiatoa, Aquifex, Hydrogenobaculum, Thermocrinis, and Hydrogenothermus. Other microorganisms include other gram-positive bacteria or proteobacteria that are obligate or facultative anaerobes as well as those that are syntrophs with other microorganisms.

In some embodiments, the isolated microorganism(s) include a methanogen. In other embodiments, the microorganism(s) include one or more from the genus Bacillus, Clostridium, Ferribacter, Gelria, Geobacillus, Methanobacter, Moorella, Thermacetogenium, Thermotoga or Pseudomonas. Alternatively, the microorganism(s) include one or more from the family Propionibacteriaceae or genus Propionibacterium. Particular microorganisms that may be isolated, alone or in combination with other microorganisms include Clostridium fervidus, Ferribacter thermoautotrophicus, Gelria glutamica, Methanobacter wolfeii, Methanobacter thermoautotrophicus, Moorella glycerini, Moorella mulderi, Thermacetogenium phaeum, Thermotoga hypogea, Thermotoga lettingae, Thermotoga subterranean, Thermotoga elfii, Thermotoga maritima, Thermotoga neapolitana, Thermotoga thermarum, and Thermotoga petrophila.

In other embodiments, the isolated microorganisms can convert native carbonaceous materials of a geologic formation and/or the hydrocarbons contained therein into hydrocarbons having a greater mol. % of hydrogen atoms, such as methane. In their native state, carbonaceous materials such as coals and oils contain complex, polymeric hydrocarbons with multiple saturated and unsaturated carbon-carbon, carbon-nitrogen, carbon-sulfur, and carbon-oxygen bonds. The hydrocarbons are also large, which as used herein refers to hydrocarbons of 20 or more carbon atoms and/or 400 or more gm/mol in molecular weight. Moreover, and as used herein, “hydrocarbon” refers to molecules containing only carbon and hydrogen atoms, optionally containing one or more nitrogen, sulfur, and oxygen atoms. The invention provides isolated microorganisms and consortia comprising them that are capable of converting the complex and/or large hydrocarbons into smaller molecules, including smaller hydrocarbons with less than 20 carbon atoms and/or 400 gm/mol molecular weight.

During conversion of complex and/or large hydrocarbons into smaller hydrocarbons, the ratio of C—C to C—H bonds is typically reduced, resulting in higher mol. % of hydrogen atoms for the product molecules because of an increase in the number of hydrogen atoms relative to the number of non-hydrogen atoms in a product molecule. For example, acetic acid has the chemical formula CH₃COOH, representing 2 carbon atoms, 2 oxygen atoms, and 4 hydrogen atoms, to give a total of 8 atoms. Since 4 of the 8 atoms are hydrogen, the mol. % of hydrogen atoms in acetic acid is: (4 Hydrogen Atoms)/(8 Total Atoms)=0.5, or 50%, by mol. (or on a molar basis). Methane has the chemical formula CH₄, representing 1 carbon atom and 4 hydrogen atoms, making a total of 5 atoms. The mol. % of hydrogen atoms in methane is (4 Hydrogen Atoms)/(5 Total Atoms)=0.8, or 80%, by mol. Thus, the conversion of acetic acid to methane increases the mol. % of hydrogen atoms from 50% to 80%. In the case of molecular hydrogen, the mol. % of hydrogen atoms is 100%. The invention provides isolated microorganisms capable of providing a net increase in the mol. % of hydrogen atoms, starting from a complex and/or larger hydrocarbon to a final smaller hydrocarbon, is from less than about 66% to 80 or 100%, from about 66% to 80 or 100%, or from about 70% to 80 or 100%.

In some cases, each step of a microorganism(s)' metabolic pathway increases the mol. % of hydrogen atoms of the resultant metabolite. For example, in a three-step metabolic pathway where: (1) a portion of the native carbonaceous material is metabolized into a phenol; (2) the phenol is metabolized into acetic acid; and (3) the acetic acid is metabolized into methane, the mol. % of hydrogen atoms increases at each step. In other cases, intermediate steps in the metabolic pathway may decrease the mol. % of hydrogen atoms. For example, another three-step metabolic pathway may include the metabolic steps of: (1) converting native carbonaceous material to acetic acid; (2) converting the acetic acid to hydrogen (H₂) and carbon dioxide (CO₂); and (3) converting the H₂ and CO₂ into methane and water. For this metabolic pathway, the mol. % of hydrogen atoms goes from 100% for H₂, to 80% for methane, which represents a decrease in the mol. % hydrogen between steps (2) and (3). However, there is still an increase in the mol. % hydrogen between the starting carbonaceous materials and the final metabolic product (i.e., methane).

Native anaerobic consortia have been collected from a variety of sub-surface formations, and studied in a controlled, low-oxygen environment to classify the functions of each consortium or subpopulation that makes up the consortia, as well as the microorganisms that make up each consortium or subpopulation. Rates of biogenic hydrocarbon production have also been compared between consortia to identify microorganisms, and combinations of consortia that are particularly effective at converting carbonaceous materials into other hydrocarbons that have higher mol. % hydrogen. Isolation of these microorganisms as consortia has led to the embodiments of the present invention, which include an isolated microbial consortia comprising a first microbial consortium capable of converting large and/or complex hydrocarbons into a product comprising one or more first intermediate hydrocarbons; a second microbial consortium, comprising one or more species of Pseudomonas or Thermotoga, capable of converting one or more of the first intermediate hydrocarbons into a product comprising one or more second intermediate hydrocarbons and one or more molecules comprising oxidized carbon; and a third microbial consortium capable of converting one or more of the second intermediate hydrocarbons into a product comprising one or more smaller hydrocarbons and water, wherein the smaller hydrocarbons have a greater mol. % hydrogen than the large and/or complex hydrocarbons.

In further embodiments, the microorganisms further comprise one or more syntrophs, which are microorganisms related by one or more syntrophic interactions. This may be viewed as a state of mutual dependence, or interdependence, among different groups of microorganisms wherein each group contains one or more member microorganisms. The member(s) of a first group may be related to member(s) of one or more other groups such that the member(s) of the first group are dependent upon member(s) one or more other groups for one or more substrates. The one or more other groups may also be dependent on the member(s) of the first group for one or more substrates.

As a first non-limiting example, two microorganisms (or two groups of microorganisms) may form a syntrophic acetate oxidation pathway, where acetate is converted to methane at an enhanced metabolic rate. A first microorganism (or group thereof) converts acetic acid and/or acetate (H₃CCOO⁻) into carbon dioxide and hydrogen, which may be rapidly metabolized by a second microorganism, like a methanogen, into methane and water. In a further non-limiting example, removal of the metabolites (e.g., hydrogen, carbon dioxide) produced by a first microorganism (or group thereof) by a second microorganism (or group thereof) prevents the metabolites from building up to a point where they can reduce metabolism and growth in the first microorganism. In turn, the first microorganism provides a steady supply of starting materials, or nutrients, to the second microorganism. This latter syntrophic interaction between the microorganisms results in the metabolic pathway that converts acetate into methane and water being favored.

Syntrophic interactions may also be formed between microorganism populations at other points in a metabolic process, and may be established between members within a consortium (i.e., an intraconsortium interaction), as well as between members of different consortia (i.e., and interconsortium interaction). For example, a syntrophic interaction may exist between acetogens, which form the acetate, and the microorganisms that oxidize the acetate into carbon dioxide and hydrogen. In metabolic processes with multiple steps, several syntrophic interactions may occur down the pathway from reactants to products. As used herein, syntrophy refers to symbiotic cooperation between two metabolically different types of microorganisms (partners) wherein they rely upon each other for degradation of a certain substrate. This often occurs through transfer of one or more metabolic intermediate(s) between the partners. For efficient cooperation, the number and volume of the metabolic intermediate(s) has to be kept low. In one non-limiting example pertinent to the present invention, syntrophs include those organisms which oxidize fermentation products from methanogens, such as propionate and butyrate, that are not utilized by the methanogens. These organisms require low concentrations of molecular hydrogen to ferment substrates to carbon dioxide, so are symbiotic with methanogens, which help maintain low molecular hydrogen levels. Without being bound by theory, and offered to improve the understanding of the invention, microorganisms from the genus of Thermoacetogenium, Syntrophobacter, Gelria, and Clostridia are believed to be syntrophs as described herein.

The anaerobic conditions used are those of less than about 200 ppm molecular oxygen in the environment of the microorganism(s). The environment may be that of a liquid or solid medium as well as the gaseous phase above the medium. In some embodiments, the conditions are determined based upon oxygen concentrations in the gaseous phase. In other embodiments, the molecular oxygen concentration may be less than about 180 ppm, less than about 160 ppm, less than about 140 ppm, less than about 120 ppm, less than about 100 ppm, less than about 80 ppm, less than about 60 ppm, less than about 40 ppm, less than about 20 ppm, or less than about 10 ppm.

In further embodiments, the molecular oxygen concentration is maintained by use of an anaerobic jar (e.g. Brewer's Gas Pak) or analogous device which uses a palladium catalyst and hydrogen to react with gaseous molecular oxygen to form water. Such a device reduces gaseous molecular oxygen in the sealed atmosphere of the device and thus reduces the oxygen concentration in the culture contained therein. Without being bound by theory, and offered to improve the understanding of the invention, use of hydrogen and a palladium catalyst to remove oxygen may be of reduced or no interest in cases of microorganisms that generate molecular hydrogen as a metabolic product because the presence of hydrogen from the device or in the culture may shift the metabolic activity of the microorganisms to other pathways, including organic acid or alcohol production as non-limiting examples. But microorganisms that utilize molecular hydrogen as a starting material may benefit from the use of such a device or culture conditions, because the presence of hydrogen in the device is less likely to shift (toward cell growth and/or proliferation) the dynamic equilibrium between reactants and products in the metabolic pathways of such microorganisms.

In alternative embodiments, the nutrient broth used in the invention may contain a reducing agent which removes oxygen from the broth. Non-limiting examples of such agents include sodium thioglycolate, DTT, and β-mercaptoethanol or other thiol containing reducing agents at appropriately low levels to prevent interference with cellular metabolism. Other additives to the solid or liquid medium include indicators, such as a dye which changes chromatographic properties between reduced and oxidized states. Non-limiting examples include resazurin (Alamar Blue), which is detectably pink (and highly fluorescent) in the oxidized resorufin form (higher oxygen concentration) and blue-ish to colorless (and non-fluorescent) in the reduced form (lower oxygen concentration); and methylene blue, which indicates the presence of oxygen via a detectably green or blue color. In some embodiments, the indicator is one that produces a signal visibly detectable to the unaided eye.

The subterranean substrate from which microorganism(s) are isolated may be from any underground formation or materials found below more than about 10 feet of surface soil, sand or rock nearest the earth's atmosphere. In some embodiments, the substrate may be about 20 feet, about 30 feet, about 40 feet, about 50 feet, about 75 feet, about 100 feet, about 125 feet, about 150 feet, about 175 feet, about 200 feet, about 225 feet, about 250 feet, about 275 feet, or about 300 feet below surface soil, sand or rock nearest the earth's atmosphere. Alternatively, the substrate may be from the same depths as described above but below the floor of a sea, ocean or other body of water, like a substrate from the depths of an underwather oil well as a non-limiting example. Non-limiting examples of the location of possible substrates include mines and wells from which fossil fuels like coal and oil have been or may be extracted. In some embodiments, the substrate is from a depth below which oxygen from the atmosphere or other environment above the formation can appreciably penetrate. In additional embodiments, the substrate may be from a bioremediation site containing a highly complex mixture of saturated aromatic and aliphatic hydrocarbons as principle components.

As used herein, “substrate” includes solids, liquids and gases, including rocks, shale, and sediment as non-limiting examples. Combinations of a solid and liquid may also be used, so long as microorganisms are present in the substrate. In some embodiments, the substrate comprises hydrocarbons. Native carbonaceous material found on earth, such as oil, coal, coke, kerogen, oil shale, tar, tar sands, anthracite, coal tar, bitumen, lignite, peat, carbonaceous shale, and sediments rich in organic matter, as well as water found with such materials or water found in geologic formations containing such materials are non-limiting samples of additional substrates of the invention. A non-limiting example of such water containing substrates is formation water from a geologic environment, including a mine or well used as a source of solid (e.g. coal), liquid (e.g. crude oil or petroleum), or gaseous (e.g. natural gas) hydrocarbon.

In the case of a liquid substrate, the microorganism containing material can be obtained and maintained under anaerobic, anoxic, or methaogenic conditions until the substrate is used in the isolation methods of the invention. The substrate, such as anaerobically or anoxically collected formation water of a subterranean site, may optionally be filtered anaerobically to collect and concentrate microorganisms contained therein. A sample of a liquid substrate, or filtrate thereof, is contacted with an appropriate culture medium and then maintained under the conditions of the instant methods.

In the case of a solid substrate, the microorganism containing material can be obtained as a core sample and optionally maintained under the conditions described herein until use in the isolation methods of the invention. For use in some embodiments, a fresh face of the solid substrate may be obtained by cutting the sample to allow collection of microorganism from the interior of the samples. Such cutting actions, as well as all manipulative acts necessary to the practice of the invention may, of course be conducting under the conditions of the invention as described herein. As a non-limiting example, the cutting (or the transfer of microorganisms) may be performed in an anaerobic chamber or “glove bag”.

Where the solid substrate is of a size small enough for direct contact with a liquid medium and subsequent culturing therein, the substrate is so used. Non-limiting examples of such substrates include sand or small pebbles or rocks as well as pulverized solid hydrocarbons (e.g. coal reduced to a size ranging from ˜5 microns (μm) to ˜100 microns).

The isolation methods of the invention may also include the further step of isolating one or more microorganisms, from a population (or coculture or consortium) of initially isolated microorganisms, to form a second culture of microorganisms. Thus where more than one microorganism is initially isolated, the methods of the invention provide for the further isolation or separation of one or more microorganisms from the initial isolate (or culture) to form a second culture.

Furthermore, the invention provides for the subsequent isolation, and optional analysis, of genetic material from cultured or isolated microorganisms for study and analysis. This further analysis may advantageously be used to identify or classify the microorganism(s) present in the isolate. The subsequent isolation of genetic material may be preceded by lysis of isolated microorganisms to form lysed cellular material from which the genetic material is obtained or analyzed. The cellular material may be optionally extracted to form a cell extract before the genetic material is obtained or analyzed.

The analysis of genetic material from isolated microorganisms may be part of a method of detecting the presence of a microorganism in the subterranean substrate from which the microorganism was obtained. Such a method may comprise, after preparing a culture of microorganism(s) as described herein, detecting the presence of one or more microorganisms in said culture. In some embodiments, the detection is of the genetic material (DNA or RNA) of the microorganism(s). Alternatively, the detection is of proteins, or portions thereof, expressed by the microorganism(s), such as by use of antibodies or other ligands (or binding partners) to the proteins or portions thereof.

In embodiments utilizing the detection of DNA, the DNA is optionally cloned into a vector and suitable host cell to amplify the amount of DNA to facilitate detection. In some embodiments, the detecting is of all or part of ribosomal DNA (rDNA), of one or more microorganisms. Alternatively, all or part of another DNA sequence unique to a microorganism may be detected. Detection may be by use of any appropriate means known to the skilled person. Non-limiting examples include restriction fragment length polymorphism (RFLP) or terminal restriction fragment length polymorphism (TRFLP); polymerase chain reaction (PCR); DNA-DNA hybridization, such as with a probe, Southern analysis, or the use of an array, microchip, bead based array, or the like; denaturing gradient gel electrophoresis (DGGE); or DNA sequencing, including sequencing of cDNA prepared from RNA as non-limiting examples.

Non-limiting examples of unique, or potentially unique sequences include those encoding ribosomal RNA (rRNA) as well as other sequences believed to be found in many if not all prokaryotes. Sequences of the fusA, ileS, lepA, leuS, pyrG, recA, recG, rplB, and rpoB genes (see Santos, S. R. and Howard Ochman. “Identification and phylogenetic sorting of bacterial lineages with universally conserved genes and proteins.” Environmental Microbiology. 2004. July(6)7:754-9) may be used as additional non-limiting examples. In some embodiments, the sequence to be detected is that of a rDNA. 16S rRNA encoding sequences may be used as a non-limiting example. Alternatively, detection is by identification of the 16S/23S rRNA intergenic spacer region size.

Where RFLP is used, the DNA may be cleaved by a plurality (from 2-5 or more) restriction enzymes to generate multiple permutations of fragment lengths for detection and analysis. As understood by the skilled person, the TRFLP is performed via gel electrophoresis to separate out individual nucleic acid lengths (such as 16S rRNA lengths as a non-limiting example) to identify or classify one or more microorganisms on the basis of the sequence lengths.

Where DNA sequencing is used, the detected sequence is compared to sequences of known microorganisms to provide a basis for identification or classification. Where PCR is used, the selected primers may be used to amplify a specific unique target sequence or be themselves complementary to unique sequences. In either instance, the amplified material (amplicon) may be detected as a basis for identification or classification of microorganism(s). Full DNA sequencing of one or more target sequences can always be conducted to confirm an identification or classification.

In embodiments utilizing the detection of RNA, the RNA is optionally amplified to facilitate detection. Amplification may be in the form of cDNA or in the form of linear RNA amplification (using a cDNA template with a suitable promoter) as known to the skilled artisan. Alternatively, reverse transcription based PCR (RT-PCR) may be used, optionally as part of quantitative PCR (QPCR) or real-time PCR. In some embodiments, the detecting is of all or part of ribosomal RNA (rRNA), of one or more microorganisms. Alternatively, all or part of another RNA sequence unique to a microorganism may be used.

Where RNA is the molecule to be detected, suitable measures known to the skilled person to reduce or prevent RNA degradation during nucleic acid material isolation and use should be taken. Use of protein denaturants, RNase inhibitors, and other means to reduce RNA degradation may be used in the practice of the invention.

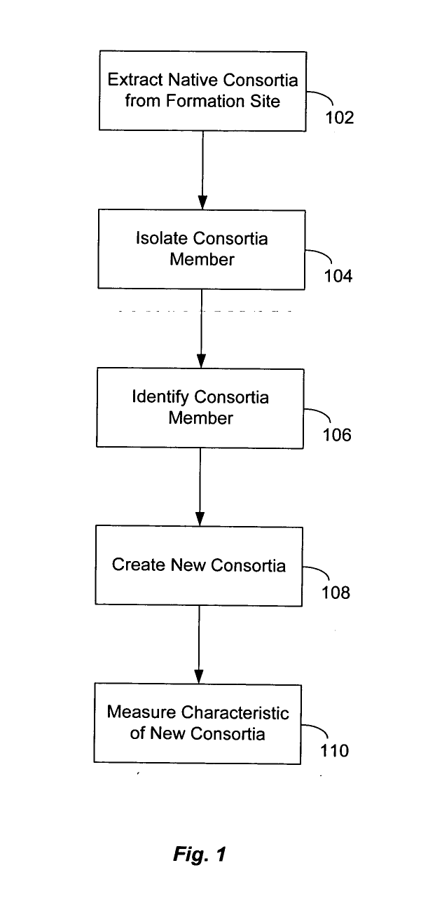

FIG. 1 shows a flowchart with method steps for making and measuring the characteristics of a consortium of microorganisms. In the embodiment shown, the method starts with extracting native microorganisms from a formation site 102. The microorganisms may be taken from solid substrate at the site and/or formation water stored in the site. Subsets and/or individual members are isolated from the extracted consortia 104 in a manner as disclosed herein. The microorganisms may also be identified 106, such as after they are isolated by methods as disclosed herein.

The method may also include the creation of new consortia 108 by combining members and/or subsets of the native consortia to form a new consortia. Genetically modified microorganisms not found in any native consortia may also be introduced. Characteristic of the new consortia may be measured 110, such as the consumption rate of carbonaceous material and/or the production rate of metabolite (e.g., methane). Measured characteristics may also involve the response of the new consortia to amendments made to the consortia's environments, such as changes in temperature, pH, oxidation potential (Eh), nutrient concentrations, salinity, metal ion concentrations, etc.

The invention also provides additional methods for obtaining nucleic acids from a hydrocarbon containing substrate. This may be performed prior to use of disclosed isolation for culture methods, optionally to identify the microorganisms in the substrate prior to the culturing of microorganisms from the substrate as described herein. The obtained nucleic acids may be used to determine whether the substrate has microorganisms of interest for subsequent culture, such as microorganisms which have not previously been isolated. The methods to obtain nucleic acids from a substrate provide the advantage of being able to perform nucleic acid based analysis for identification or classification of microorganisms without the need to culture microorganisms as a precondition.

Thus the invention includes a method of eluting or desorbing microorganisms (as well as any cell) adsorbed to a carbonaceous substrate followed by preparation of genetic material from the microorganisms. Alternatively, the method may be used to obtain any cell-free genetic material that is directly present on or in a carbonaceous substrate. Nucleic acids present in cells associated with a carbonaceous substrate may be considered to be indirectly associated with the same substrate. In some embodiments, these methods need not be performed under anaerobic, anoxic, or methanogenic conditions. The method comprises contacting the microorganisms, or cell-free nucleic acids, associated with a carbonaceous substrate with a solution containing one or more anions and a zwitterionic detergent to form a mixture, and then mechanically agitating said mixture to elute the nucleic acids from the substrate into the solution. Of course it need not be known in advance whether a carbonaceous substrate has associated microorganisms, or cell-free nucleic acids. Thus the method may also be practiced with a substrate that is merely suspected of having associated microorganisms, or cell-free nucleic acids, or a substrate for which a curiosity exists with respect to whether it has such associated materials. Similarly, and while the invention is described mainly with respect to carbonaceous substrates from an underground (or underwater) environment (as described above), the method may be practiced with any carbonaceous substrate, including manmade materials. Non-limiting examples include activated carbon and charcoal. In other embodiments of the invention, the substrate is coal, raw or processed.

While the discussion above and below are in relation to microorganisms associated, absorbed, or adsorbed to a carbonaceous substrate, the methods may also be equally applied to any cell. Thus cells of a multicellular organism or animal, as well as nucleic acids therefrom, may also be eluted or desorbed by the disclosed methods.

The associated microorganisms or cell-free nucleic acids may be those that are already present with a carbonaceous substrate when it is obtained from a geological formation (e.g. subterranean location) as described herein. Where the cell(s) of a microorganism or other organism is the case, the invention provides for the lysis of the cell(s) associated with the substrate to release the cellular contents, including nucleic acids. This lysis may be in the presence of the carbonaceous substrate, in which case the nucleic acids are allowed to come into contact with the substrate. This facilitates the recovery of those released nucleic acids from the substrate. Alternatively, the lysis may be after the cells have been isolated from the substrate.

Lysis may be by any means known in the art. Non-limiting examples include the use of detergents, chaotropic agents, and/or organic solvents which disrupt cellular integrity. SDS, alone or in combination with an agent like guanidinium thiocyanate or DTT or urea, as well as DNAzol (a detergent and guanidinium thiocyanate combination) are additional non-limiting examples along with phenol and/or chloroform as examples of organic solvents. As an alternative after lysis of the cells, the cellular contents may be extracted and then nucleic acids isolated therefrom.

In other embodiments, lysis may be mediated by use of a proteolytic agent, such as, but not limited to, proteinase K as known in the art. The use of such an agent may be instead of the above described use of detergents, chaotropic agents, and organic solvents, or in combination with a detergent and/or chaotropic agent as described above.

The nucleic acids may be DNA or RNA of any type associated with a carbonaceous substrate. Where RNA is to be obtained, the method may be practiced with appropriate inhibitors of RNA degradation. In some embodiments, the nucleic acids are those from one or more microorganism as described within the present disclosure.

The method is practiced with the use of anions, which as used herein include pyrophosphate (or other polyphosphates) as well as other anions. This reflects a discovery that the association of cells (e.g. microorganisms) and cell-free nucleic acids to a carbonaceous substrate may be based in part on charge interactions. Other non-limiting examples for use in the method include nucleic acid polymers (like RNA or DNA), such as that from a non-prokaryotic or non-archaebacterial source. Yeast and higher eukaryotic nucleic acids may also be used in some embodiments. Alternatives include homo or other synthetic polymers, such as polyA, polyG, polyC, polyU, polyl, poly(dA), poly(dT), poly(dG), poly(dC), or oligo versions thereof as non-limiting examples. Thus the use of a wide variety of polyanions is contemplated for the practice of the invention.

A variety of zwitterionic detergents known to the skilled artisan may be used in the practice of the invention. Non-limiting examples include zwittergent (n-Decyl-N,N-dimethyl-3-ammonio-1-propanesulfonate), CHAPS, and CHAPSO. In some embodiments, the detergent is non-denaturing.

Optionally, the method may be practiced in the presence of one or more chelating agents, such as chelators of multiple divalent cations (e.g. EDTA) and relatively specific chelators (e.g. EGTA to chelate calcium cations) as non-limiting examples. In some embodiments, the inclusion of a chelator to disrupt interactions mediated by calcium cations is used advantageously in the practice of the invention.

After contacting a carbonaceous substrate with both anions and zwitterionic detergent as described herein, the mixture is agitated in some embodiments. The agitation may be vigorous, such as with the use of blending or pulse blending. Without being bound by theory, and offered to improve the understanding of the invention, both the treatment with an anion and detergent as well as the agitation are believed to aid in the release of cells and/or cell-free nucleic acids from a carbonaceous substrate. Following such release, the carbonaceous substrate may be separated from the solution containing the cells or nucleic acids. In some embodiments, the separation is performed by filtration. In others, centrifugation may be used.

The resultant solution may then be treated for the isolation of the nucleic acid material. Where cells are first obtained, the cells may be lysed as described above before nucleic acids are isolated. The isolation of nucleic acids from solution may be by any means known to the skilled person, such as, but not limited to, precipitation, affinity chromatography, gel separation, and the like. In preferred situations, the nucleic acids contain detectable sequences as described within the present disclosure.

While the present invention has been mainly described in relation to methods and processes, the invention also encompasses the embodiments of the disclosed methods and processes as systems comprising one or more device or apparatus for practicing the invention. Thus in some embodiments, a system comprising one or more devices to perform one or more of the methods or processes disclosed herein is provided. In other embodiments, a system comprising more than one device, each performing one or more of the acts of the methods or processes disclosed herein, is provided. Non-limiting examples of devices in the systems of the invention include devices to culture or otherwise maintain microorganisms ex situ and devices to analyze nucleic acids as described herein.

Having now generally described the invention, the same will be more readily understood through reference to the following examples which are provided by way of illustration, and are not intended to be limiting of the present invention, unless specified.

### EXAMPLES

### Example 1

### Isolation of Microorganisms

Native anaerobic microorganisms have been collected from a variety of sub-surface formations, and studied in a controlled, low-oxygen environment to classify the functions of each consortium that make up the consortia, as well as the microorganisms that make up each consortium. Rates of biogenic hydrocarbon production have also been compared between consortia to identify microorganisms, and combinations of microorganisms that are particularly effective at converting carbonaceous materials into other hydrocarbons that have higher mol. % hydrogen. Isolation of these microorganisms as consortia has led to embodiments of the present invention.

### Example 2

### DNA Purification Protocol 1

The following steps may be used to purify DNA from cells associated with a coal containing sample. The solutions needed are as follows (where PPi is pyrophosphate):

| | Solution B | |
|---|---|---|
| Buffer A: | (per sample): | Buffer C (per sample): |
| 100 mM Tris-HCL | 20 μl RNA | 100 mM Tris-HCL pH 7.0 |
| pH 7.0 | | |
| 10 μM Zwittergent | 5 μl of Poly(A) | 1 μM Zwittergent |
| 50 mM EGTA | 50 μl of 10% PPi | 1 mM EGTA |
| filter and | make anaerobic | 1 mM EDTA |
| make anaerobic | | 30 μl lysozyme (100 mg/ml) |
| | | 30 μl mutanolysin (5 KU/ml) |
| | | filter and |
| | | make anaerobic |

A non-limiting protocol is as follows:

1) Aseptically transfer anaerobic buffer A to a slurry bottle containing coal particles to be extracted, mix, and add anaerobic solution B; mix and chill for 10 minutes. Without being bound by theory, and offered to improve the understanding of the invention, this is believed to at least begin dissociation of cells (as well as any cell-free nucleic acids) from the coal particles.

2) Filter the mixture to remove the particles.

3) Spin down the solids (including the cells) and gently remove the supernatant, which is discarded. Optionally, the supernatant can be treated as a source of cell free nucleic acids.

4) To the solids, add freshly prepared anaerobic buffer C, mix, and agitate at 37° C. for 1 hour.

5) Remove supernatant to new tube, filter if necessary.

6) Precipitate nucleic acid material, remove supernatant, and wash with cold 70% ethanol before pelleting again and removal of supernatant. Allow pellet to dry before rehydrating in buffered solution.

The above may be conducted in the presence of RNase inhibitors (and DEPC treated materials) as needed.

### Example 3

### DNA Purification Protocol 2

The following steps may be used to purify DNA from cells associated with a coal containing sample with blending. The solutions needed are as provided above. The non-limiting protocol is as follows:

1) Aseptically transfer anaerobic buffer A to a slurry bottle containing coal particles to be extracted, mix, and add anaerobic solution B; mix and chill for 10 minutes.

2) Anaerobically remove the slurry contents into a sterile anaerobic 30 ml blender cup. Blend the sample for one minute using a Waring blender. Filter the blended sample through a sterile 20 μm filter and retain the filtrate.

2) Spin down the solids in the filtrate and gently remove and discard supernatant.

3) Add freshly prepared anaerobic buffer C, mix, and agitate at 37° C. for 1 hour.

4) Add 5 ml of Dnazol (or alternative solution containing a chaotropic agent) and 60 μl of freshly prepared, filtered proteinase K (20 mg/ml stock), mix and let sit at RT for 1-24 hours.

Mechanically disrupt the mixture, such as by bead-beating; pellet and remove supernatant to new tube, filter if necessary.

5) Precipitate nucleic acid material, remove supernatant, and wash with cold 70% ethanol before pelleting again and removal of supernatant. Allow pellet to dry before rehydrating in buffered solution.

The above may be conducted in the presence of RNase inhibitors (and DEPC treated materials) as needed.

Having described several embodiments, it will be recognized by those of skill in the art that various modifications, alternative constructions, and equivalents may be used without departing from the spirit of the invention. Additionally, a number of well known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the electrode” includes reference to one or more electrodes and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A method of eluting or desorbing nucleic acids adsorbed to a carbonaceous substrate, said method comprising
contacting cells, associated with said substrate and containing said nucleic acids, with a solution containing one or more anions and a zwitterionic detergent to form a mixture, and
mechanically agitating said mixture to elute said cells from said substrate into said solution.

2. The method of claim 1, further comprising separating said carbonaceous substrate from said solution, optionally by filtering said substrate from said solution.

3. The method of claim 1 wherein said nucleic acid is DNA, optionally encoding 16S rRNA, or said substrate is coal.

4. The method of claim 1 wherein said one or more anions comprises pyrophosphate or non-prokaryotic RNA.

5. The method of claim 1 wherein said zwitterionic detergent is n-decyl-N,N-dimethyl-3-ammonio-1-propanesulfonate.

6. The method of claim 1 wherein said agitating is with pulse blending of said mixture.

7. The method of claim 1, further comprising isolating said nucleic acid from said solution.

8. The method of claim 1 wherein the nucleic acid is from a microorganism selected from one or more from the genera Bacillus, Clostridium, Ferribacter, Gelria, Geobacillus, Methanobacter, Moorella, Thermacetogenium, Propionbacterieae, Pseudomonas or Thermotoga.

9. A method of preparing a microbial community of microorganisms, said method comprising
obtaining an anaerobic subterranean substrate comprising viable microorganisms; and
maintaining said substrate under anaerobic conditions to form an ex situ microbial community.

10. The method of claim 9, wherein the subterranean substrate comprises coal, oil, kerogen, peat, lignite, oil shale, tar sands, bitumen, water, and tar.

11. The method of claim 9, further comprising isolating or separating
1) one or more microorganisms from said community, or alternatively
2) lyse cellular material from said one or more microorganisms, optionally as a cell extract.

12. The method of claim 11, wherein said one or more microorganisms comprises a methanogen, or wherein said maintaining comprises the use of sterile anaerobic buffer solutions.

13. The method of claim 9, wherein said microorganisms comprise one or more from the genera Bacillus, Clostridium, Ferribacter, Gelria, Geobacillus, Methanobacter, Moorella, Thermacetogenium, Propionbacterieae, Pseudomonas or Thermotoga.

14. The method of claim 4, wherein said microorganisms further comprise one or more syntrophs.

15. The method of claim 5, wherein said microorganisms comprise one or more selected from Clostridium fervidus, Ferribacter thermoautotrophicus, Gelria glutamica, Methanobacter wolfeii, Methanobacter thermoautotrophicus, Moorella glycerine, Moorella mulderi, Thermacetogenium phaeum, Thermotoga hypogea, Thermotoga lettingae, Thermotoga subterranean, Thermotoga elfii, Thermotoga maritima, Thermotoga neapolitana, Thermotoga thermarum, and Thermotoga petrophila.

16. A method of detecting the presence of a microorganism in an anaerobic subterranean substrate, said method comprising
preparing microorganisms according to claim 9, and
detecting the presence of one or more microorganisms in said preparation.

17. The method of claim 16 wherein said detecting is of rDNA, optionally encoding 16S rRNA, of one or more microorganisms.

18. The method of claim 16 wherein said detecting is by restriction fragment length polymorphism (RFLP), optionally terminal restriction fragment length polymorphism (TRFLP); or by PCR; or by DNA-DNA hybridization; or by denaturing gradient gel electrophoresis (DGGE); or by DNA sequencing;

19. The method of claim 16 wherein said detecting is of rRNA, optionally by complementary DNA (cDNA) sequencing or detection; or by reverse transcription-PCR (RT-PCR), optionally as part of quantitative or real-time PCR; or by identification of 16S/23S rRNA intergenic spacer regions size.
